(19) United States
(12) Patent Application Publication
(10) Pub. No.: US 2024/0013245 A1
(43) Pub. Date: Jan. 11, 2024

(54) SYSTEM AND METHOD FOR PROVIDING DRUG AND MEDICAL PRICING INFORMATION

(71) Applicants: David A. Harrell, Naples, FL (US); Karthik Kalyana Sundaram, Frisco, TX (US); Neil H. Bajaj, Glen Allen, VA (US)

(72) Inventors: David A. Harrell, Naples, FL (US); Karthik Kalyana Sundaram, Frisco, TX (US); Neil H. Bajaj, Glen Allen, VA (US)

(21) Appl. No.: 17/985,230

(22) Filed: Nov. 11, 2022

Related U.S. Application Data
(63) Continuation-in-part of application No. 17/671,238, filed on Feb. 14, 2022.
Application No. 17/671,238 is a continuation-in-part of application No. 17/174,538, filed on Feb. 12, 2021.

(51) Int. Cl. G06Q 30/0201; G16H 20/10; G06Q 30/0601

(52) U.S. Cl. CPC G06Q 30/0206; G16H 20/10; G06Q 30/0613

(57) ABSTRACT

A system and method for determining a lowest purchase method and/or combination of purchase methods for purchasing one or more medical products, such as prescription drugs, and/or medical services, such as medical, dental, or veterinary procedures, wherein a lowest out-of-pocket expense to a user is determined based on various pricing variables, insurance reimbursement variables, and available discount programs obtained in-part from third-party data providers and third-party aggregators.

### CROSS-REFERENCE TO RELATED APPLICATIONS

This application is a continuation-in-part of U.S. patent application Ser. No. 17/671,238 filed on Feb. 14, 2022, which is currently pending and is a continuation-in-part of U.S. patent application Ser. No. 17/174,538 filed on Feb. 12, 2021, which is currently pending. The patent applications identified above are incorporated herein by reference in their entirety to provide continuity of disclosure.

### FIELD OF THE INVENTION

This invention relates to methods for searching and comparing prices for medications and medical products and services, and more particularly, a system and method for determining a lowest purchase method and/or combination of purchase methods for purchasing one or more medical products, such as drug prescriptions, wherein a lowest amount of costs to a user is determined based on various pricing, reimbursement variables, and available discounts; or one or more medical services, such as a medical procedure, wherein a lowest out-of-pocket expense to a patient is determined based on various pricing variables, discount and assistance programs and insurance reimbursement variables.

### BACKGROUND OF THE INVENTION

Conventional methods for gathering and reporting prices relating to medical products or services are based on conventional aggregating and reporting methods. For example, conventional pharmaceutical pricing aggregators operate by obtaining drug pricing information from one or more pharmacy benefit managers (“PBM”), which is then simply passed on to users looking for a lowest offered “cash” price. For example, a conventional system may obtain, calculate, and/or estimate drug prices that are available under contracts or agreements between PBMs' various pharmacies, which is information typically not available to average consumers.

In response to a request for a price of a specific drug, conventional systems will display relevant “cash” prices paid directly by the user for each pharmacy chain and/or display prices found in a particular geographic area. The users can compare prices for a particular drug and determine which pharmacy they prefer to purchase the drug from.

Conventional drug pricing methods concentrate on a lowest direct “cash” price, however, the cheapest direct price of a prescription may not equate to the lowest out-of-pocket expense for a user. Conventional systems fail to take many factors and variables, such as insurance coverage and available copay discounts, into account that affect a final amount of “cash” a user pays out-of-pocket at the register. For example, a conventional drug pricing method may find a cheapest price for a prescription but a higher price at another pharmacy may be a better deal for a user based on the user's insurance, co-pays, reimbursement sources, and other variables. Without this additional information and an analysis of that information, a user may mistakenly end up purchasing a prescription for a price believing they are receiving the best deal available to him or her, when the purchased prescription is actually costing them more than if purchased using alternative methods.

This same problem also exists for pricing related to medical services provided by doctors, dentists, veterinarians, and other providers wherein pricing and reimbursement plans are commonly withheld from the general public, similar to PBMs.

Therefore, a need exists for a system and method for determining a lowest purchase method and/or combination of purchase methods for purchasing one or more medicines, medical products or medical services wherein a lowest out-of-pocket expense to a user is determined based on various pricing variable and reimbursement variables.

### SUMMARY OF THE INVENTION

The primary object of the present invention is to provide a system and method for determining a lowest purchase method and/or combination of purchase methods for purchasing one or more medicines, medical products or medical services wherein a lowest out-of-pocket costs to a user is determined based on various pricing variable and reimbursement variables.

The present invention fulfills the above and other objects by providing a system for determining a best purchase method wherein a combination of pricing variables and/or reimbursement variables are examined, such as cash, insurance reimbursement, purchasing a generic version vs. a name brand version, quantity of the prescription, pharmacy benefit managers' (PBMs) pricing information, manufacturer's copay discount programs, patient assistance programs (PAPs), medication assistance programs (MAPs), direct-to-consumer pricing, self-funded programs, income status of the patient, the 340BDrug Pricing Program of the Public Health Services Act, third-party pricing aggregators, manufacturers' coupons, geographic location of a user/purchaser, and so forth, and to determine how each pricing variable and/or insurance reimbursement variable work together or independently to offset a price of a desired medical product or medical service for a user, such as a patient or caregiver. By accessing data through private and public pricing databases, the system and method of the present invention is capable of comparing and contrasting a plurality of variables to determine the lowest out of pocket scenario for that specific user.

Third-party pricing aggregators may include conventional prescription discount programs and online price comparison tools, wherein user input and pricing variables are used to query third-party search engines for results to be presented to the user.

An additional element of the present invention also compromises a reminder system for users to refill their medications. This includes the inclusion of the users' specific medication, dose, and prescription timelines. Reminders preferably include a coupon for a pharmacy refilling the prescription confirming the purchase method providing the lowest-out-of-pocket purchase method and/or a coupon for a new pharmacy if a lower purchase method is determined at a new pharmacy prior to a date for re-filling the prescription.

The reminder system may also be used to send or push reminder notifications reminding a user or customer to schedule an appointment or of a previously scheduled appointment at a medical provider, such as a doctor, dentist, veterinarian, and so forth.

The system and method of the present invention may be used to aggregate and present quality ratings, reviews, complaints, and so forth to present to a user in addition to pricing information so a user may make as an informed decision as possible regarding the medical service or medical product being purchased.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a schematic diagram showing an online environment in which the system and method of the present invention operates;

FIG. 2 is a flow chart showing steps for carrying out the system and method of the present invention wherein steps for determining a lowest out of pocket cost for a user are illustrated;

FIG. 3 is a flow chart showing steps for carrying out the system and method of the present invention wherein steps for monitoring a user's user of a specific drug are illustrated; and

FIG. 4 is a flow chart showing an exemplary pathway for information to flow through the system and method of the present invention.

### DESCRIPTION OF THE PREFERRED EMBODIMENTS

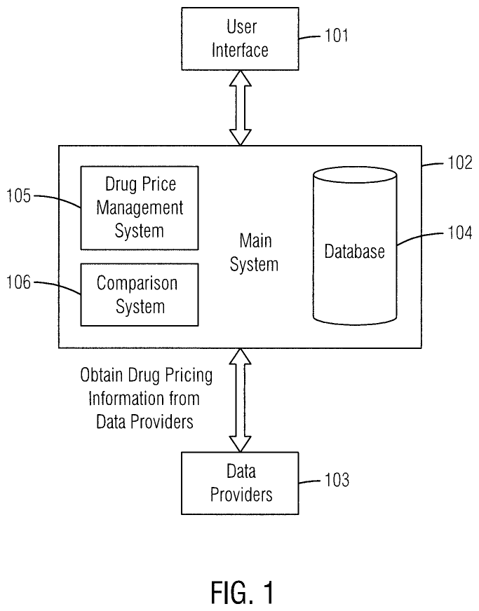

With reference to FIG. 1, an example data flow diagram for providing medical pricing information for medical services and/or medical products from multiple data providers to a user interface is illustrated. The method and system of the present invention is accessed online via a user interface 101 that is presented on an online site accessed using an electronic device capable of accessing a software application of the present invention through a provider network 102 having online access to a plurality of data providers 103.

A user, such as a patient or caregiver acting as a retail purchaser of a prescription drug, may access the provider network 102 over the Internet or app and user interface presented on the user's electronic device, which may be any suitable computing device, such as a smart phone, a tablet, a laptop, and so forth. The provider network 102 comprises at least one database 104, connected to a medical price management system 105 or module and a comparison system 106 or module for different price and payment scenarios and/or combinations.

The information obtained, analyzed, and stored within the provider network 102 is obtained from third party data providers 103, which may comprise medical providers, doctors, physician assistants, dentists, specialists, veterinarians, drug manufacturers, formulary data, PBMs, pharmacies, health insurance providers, government entities, Medicaid, Medicare, and so forth. The information obtained, analyzed, and stored within the provider network 102 comprises pricing variables and reimbursement variables provided by the data providers 103. Pricing and reimbursement variables may comprise variables relating to a medical provider's published fees, PBM data, geographic location, price lists, cash payments, insurance reimbursement, purchasing a generic version vs. a name brand version, quantity of a prescription, manufacturers' coupons, geographic location of the user and so forth.

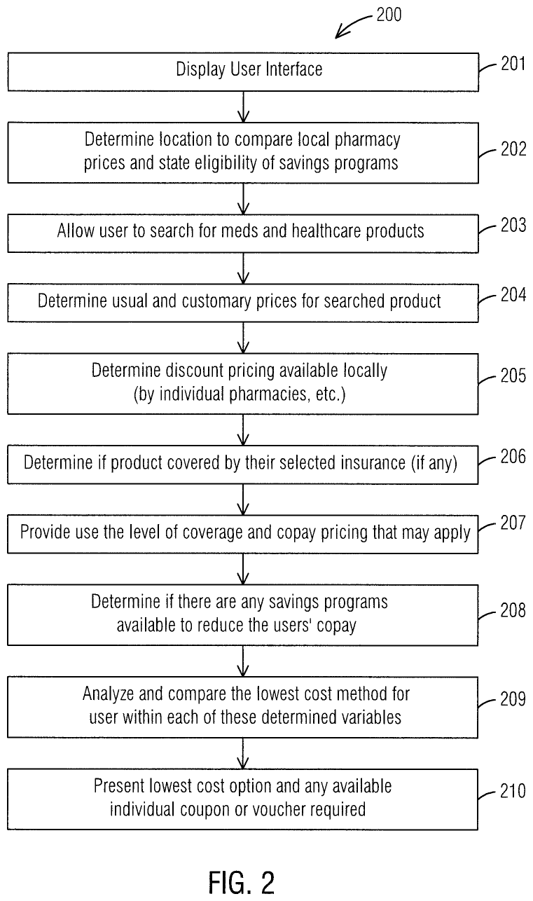

With reference to FIG. 2, a flow chart showing steps for carrying out the system and method of the present invention wherein steps for determining a lowest out of pocket cost for a user is illustrated. First, a graphical user interface is presented on a user's electronic device 201 that provides the user with access to an online site capable of accessing a software application of the present invention through a provider network having online access to a plurality of third-party data providers, as illustrated in FIG. 1. A user may be a patient, a parent or guardian of a patient, a caregiver, cashier, pharmacist, or other individual purchasing medical services and/or medical products using the system and method of the present invention and or assisting with same. The graphical user interface provides capability for a user to enter information relating to a medical service and/or medical product, such as a prescribed drug the user wishes to search after which, a lowest out-of-pocket price for the user based on a plurality of variables provided by the user and third-party data providers. The graphical user interface may be presented on an electronic device, such as a smart phone, or on a point-of-sale (POS) system when the invention is being used by a retail worker or other individual assisting a purchaser. Third party data providers may comprise medical providers, drug manufacturers, formulary data, PBMs, pharmacies, health insurance providers, government entities and so forth, that provide data relating to pricing and reimbursement variables, may include variables relating to PBM data, geographic location, cash, insurance reimbursement, purchasing a generic version vs. a name brand version, quantity of the prescription, manufacturers' coupons, location of patient and so forth.

Next, a geographical location of the user is determined using location services on the user's electronic device or from the user manually entering his or her location into the graphical user interface 202. Then, the user may search for one or more medical services and/or medical products using the graphical user interface 203. Next, the determined location is used to locate medical service providers and/or medical product providers, such as retail pharmacies, within a specified distance from the user's determined location and customary prices for searches of medical services and/or medical products are obtained 204.

Then, availability of discount pricing is determined at the located medical service providers and/or medical product providers 205. This may also comprise determining available savings programs in the specified geographic area. However, first eligibility requirements relating to any relevant savings programs, insurance coverage, etc. are examined to determine the patient's eligibility in same. Next, any insurance coverage for the medical service and/or medical product is determined 206 and a real-time level of coverage and copay pricing is provided to the user via the graphical interface 207. Then, availability of any savings programs relating to the user's determined co-pay is determined and provided to the user 208. Next, the lowest purchase method for the user within each determined variable is determined 209. Finally, the lowest purchase method for the user is presented on a coupon or voucher via the graphical interface 210.

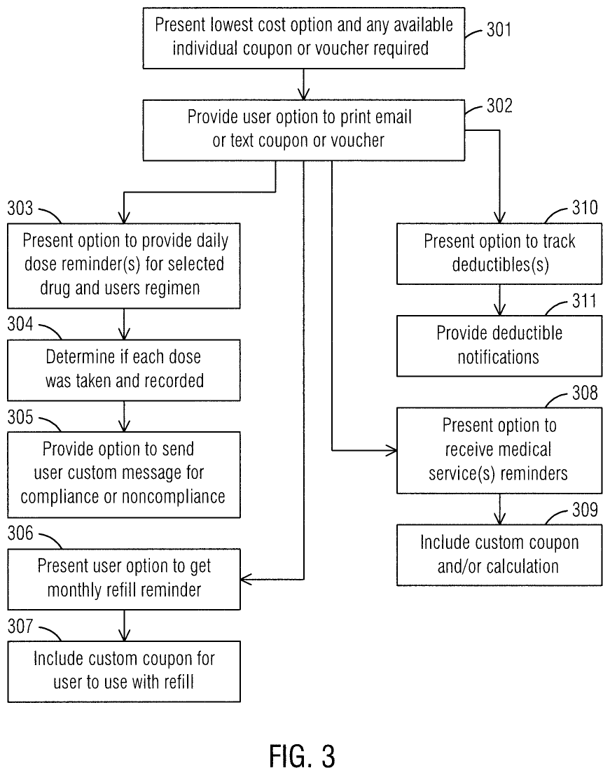

With reference to FIG. 3, a flow chart showing steps for carrying out the system and method of the present invention wherein steps for monitoring a user's user of a specific drug is illustrated. First, the lowest purchase method for the user is presented on a coupon or voucher via the graphical interface 301, as described in FIG. 2. Then, the user is provided with capability to print, email or text the coupon or voucher 302.

Next, the user is provided with capability to select reminders for administering the drug 303 to be delivered via the service provider network illustrated in FIG. 1. The user is then provided with subsequent reminders to take required doses at specified times. Next, the user confirms if he or she took the required dose using the graphical interface 304. Then, the user is sent a message confirming compliance or non-compliance 305.

The user may also be provided with capability to select reminders for refiling the prescription 306. Future reminders are delivered to the user via the service provider network with a coupon or voucher for the drug to be re-filled 307. Prescription reminders may comprise inclusion of a user's specific medication, dose, and prescription timelines. Reminders preferably include a coupon for a pharmacy refilling the prescription confirming the purchase method and thus, the lowest-out-of-pocket purchase method for the patient and/or a coupon for a new pharmacy if a lower purchase method is determined at a new pharmacy prior to a date for re-filling the prescription.

In addition, the user may be provided with capability to select reminders for future medical services, such as follow/up appointments, procedure dates, and so forth 308. Future reminders may be delivered to the user via the service provider network with a coupon or voucher for the medical service 309 and/or a real time out of pocket cost for the service.

The user may also be provided with real time deductible tracking to allow a user to determine when one or more deductibles have been met 310. Future reminders may be delivered to the user via the service provider network with expiration reminders for any met deductibles 311. The user's benefits may be tracked, searched, aggregated, and analyzed by organization and/or group health plan.

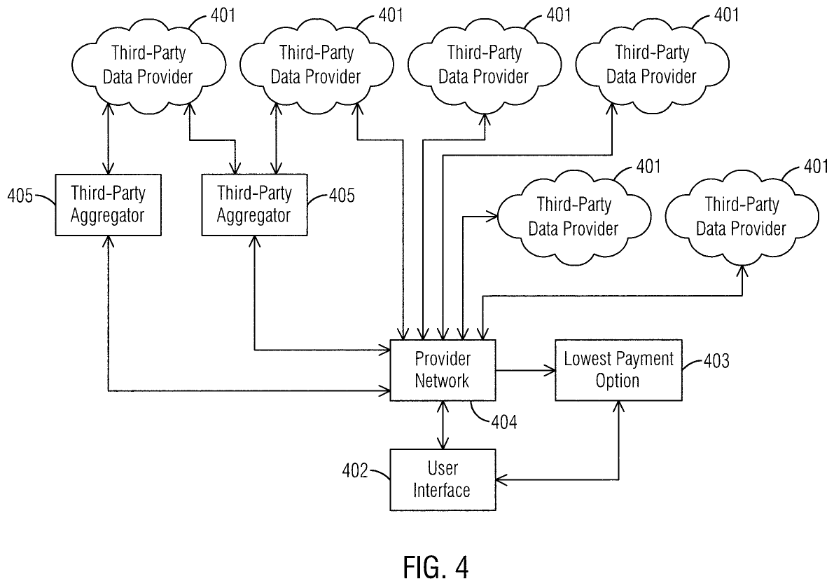

Finally, with reference FIG. 4, a flow chart showing an exemplary pathway for information to flow through the system and method of the present invention is illustrated. A lowest out-of-pocket price and purchase method is determined for a user based on a plurality of variables provided by third-party data providers 401 based on input entered into a user interface 402. The variables are analyzed to determine a best purchase method or combination of purchase methods for acquiring the best purchase price or lowest payment option 403.

The variables that are obtained, analyzed, and stored within a provider network 404 comprises pricing variables and reimbursement variables provided by the third-party data providers 401. A combination of pricing variables and/or reimbursement variables are examined, such as cash, retail, insurance reimbursement, copays, coupon copays, purchasing a generic version vs. a name brand version, quantity of the prescription, pharmacy benefit managers' (PBMs) pricing information, direct pharmacy pricing, manufacturer's copay coupon discounts, patient assistance programs (PAPs), medication assistance programs (MAPs), direct-to-consumer pricing, self-funded programs, income status of the patient, assistance programs, the 340BDrug Pricing Program of the Public Health Services Act, grants, foundation assistance, third-party pricing aggregators 405, manufacturers' coupons, online pricing, foreign imports, geographic location of a user, and so forth, and to determine how each pricing variable and/or insurance reimbursement variable work together or independently to offset a price of a desired medical product or medical service for a user. By accessing data through private and public pricing databases, the system and method of the present invention is capable of comparing and contrasting a plurality of variables to determine the lowest out of pocket scenario or purchase price for that specific user.

The system and method of the present invention may also generate and present alternative medicines to a user that. The alternative medicines would be presented with a lowest purchase price for each alternative medicine that is determined using the system and method of the present invention. If the user decides that an alternative drug or prescription would be a cheaper alternative, then the user may request a prescription change from his or her doctor. For example, the method and system of the present invention may search medicines that are comparable to a user's current prescription and suggest one or more alternative medicines that are less expensive, but still provide the same level of treatment, such as various drugs used to lower blood pressure.

Third-party aggregators 405 may include conventional prescription discount programs and online price comparison tools, wherein user input and pricing variables are used to query third-party search engines for results to be presented to the user and/or compared to, thereby providing greater access to third-party data providers 401.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

Having thus described my invention, I claim:

1. A system for determining a lowest purchase price available to a specific user for a medication, medical service or medical product, said system comprising:
a graphical user interface provided by a service provider network and connected for electronic communication through a networked communications system over the Internet;
said graphical user interface being accessible via a user's electronic device over the Internet;
said service provider network being connected to at least one database for storing information, a management system for managing inquires and data, and a comparison system for comparing a plurality of third-party data obtained from third-party data providers;
third-party data being selected based on a plurality of search parameters; and
said third-party data being multiple pricing sources, insurance reimbursement variables and any discounts or coupons that affect a determination of a final lowest purchase price for the user.

2. The system of claim 1 wherein:
said graphical user interface provides capability for a user to enter information relating to a medication the user wishes to search.

3. The system of claim 1 wherein:
said graphical user interface provides capability for a user to enter information relating to a desired medical product the user wishes to search.

4. The system of claim 2 wherein:
said graphical user interface provides capability for a user to enter information relating to a desired medical procedure the user wishes to search.

5. The system of claim 1 wherein:
said third party data providers comprise at least one drug or medical manufacturer, at least one retail pharmacy or healthcare provider, at least one PBM or other pricing partner, and at least one health insurance provider unless user does not have prescription insurance.

4. The system of claim 1 wherein:
Determining a lowest cost available for a specific user by comparing pharmacy benefit managers (PBMs) pricing versus other pricing sources, including but not limited to direct pricing from manufacturers, direct pricing from retailers, direct pricing discount drug card vendors and other non-PBM sources to display the lowest cost per user.

5. The system of claim 1 further compromising:
Comparing and determining a lowest purchase price available to a specific user for a medication, medical service or medical product within a specific retailer, healthcare provider or other purchase channel.

6. The system of claim 1 further compromising:
comparing and determining a lowest purchase price available to a specific user for a medication, medical service or medical product within multiple retailers, healthcare provider or other purchase channels.

9. The system of claim 1 wherein:
a determination being made of a lowest cost available for a specific user by comparing pharmacy benefit managers pricing versus non-pharmacy benefit managers pricing sources.

10. The system of claim 1 further comprising:
a determination being made of a lowest purchase price available to a specific user for a medication, medical service, or medical product within a specific retailer, healthcare provider, or other purchase channel.

11. The system of claim 1 further comprising:
a determination being made of a lowest cost available to a specific user for a medication, medical service, or medical product within multiple retailers, healthcare providers, or other purchase channels.

12. The system of claim 1 further comprising:
a determination of user's geographical location being determined using the user's electronic device.

13. The system of claim 12 further comprising:
a determination of a plurality of healthcare providers and/or pharmacies within a specified distance from the user's determined geographic location; and
a determination of prices for a searched medication or medical product or medical service at the plurality of located retail pharmacies and/or healthcare providers offering the medical service.

14. The system of claim 12 further comprising:
a determination of a plurality of medical service provider within a specified distance from the user's determined geographic location; and
a determination of prices for a searched medical service at the plurality of located medical providers.

15. The system of claim 13 further comprising:
a determination of discount pricing being available at one or more of the is determined at the plurality of located retail pharmacies.

16. The system of claim 14 further comprising:
a determination of discount pricing being available at at least one medical service provider.

17. The system of claim 12 further comprising:
a determination of available savings programs in a specified geographic area being made.

18. The system of claim 13 further comprising:
a determination of user's available insurance reimbursement for the searched medication being made, such as level of coverage or real time benefits check of specific user coverage.

19. The system of claim 14 further comprising:
a determination of user's available insurance reimbursement for the searched medical service being made.

20. The system of claim 13 further comprising:
a determination of a lowest purchase method for the user being determined; and
the determined lowest purchase method for the user being presented via the graphical interface.

21. The system of claim 14 further comprising:
a determination of a lowest purchase method for the user being determined; and
the determined lowest purchase method for the user being presented via the graphical interface.

22. The system of claim 1 further comprising:
a lowest purchase method for the user being presented a coupon or discount applied via the graphical interface.

23. The system of claim 1 wherein:
said selected third-party data is obtained through one or more third-party aggregators.

24. The system of claim 1 wherein:
said selected third-party data comprises a cash price, a retail price, insurance reimbursement information, available payment assistance and discount programs, and direct pricing.

25. The system of claim 23 wherein:
said selected third-party data comprises a cash price, a retail price, insurance reimbursement information, available payment assistance and discount programs, and direct pricing.

26. The system of claim 1 wherein:
said selected third-party data comprises a cash price, a retail price, insurance reimbursement information, available payment assistance and discount programs, and direct pricing.

27. A system for determining a lowest purchase price available to a specific user for a medication, medical service or medical product, said system comprising:
a graphical user interface provided by a service provider network and connected for electronic communication through a networked communications system over the Internet;
said graphical user interface being accessible via a user's electronic device over the Internet;
said service provider network being connected to at least one database for storing information, a management system for managing inquires and data, and a comparison system for comparing a plurality of said data;
said data being selected based on a plurality of search parameters; and
said data being compiled from multiple pricing sources, insurance reimbursement variables and any discounts or coupons that affect a determination of a final lowest purchase price for the user.
